(19) United States
(12) Patent Application Publication
(10) Pub. No.: US 2026/0026900 A1
(43) Pub. Date: Jan. 29, 2026

(54) Surgical System for Minimally Invasive Robotic Surgery

(71) Applicant: Deutsches Zentrum für Luft- und Raumfahrt e.V., Bonn (DE)

(72) Inventors: Christopher Schlenk, München (DE); Julian Klodmann, München (DE); Alexander Kolb, Oberpfaffenhofen-Wessling (DE); Florian Steidle, Oberpfaffenhofen-Wessling (DE)

(21) Appl. No.: 18/997,321

(22) PCT Filed: Jul. 21, 2023
(86) PCT No.: PCT/EP2023/070300

§ 371 Date: Jan. 21, 2025

(30) Foreign Application Priority Data
Jul. 22, 2022 (DE) 10 2022 118 388.2

(51) Int. Cl. A61B 34/30; A61B 1/00; A61B 90/00; A61B 90/57

(52) U.S. Cl. CPC A61B 34/30; A61B 90/03; A61B 1/00066; A61B 2034/301; A61B 2034/302; A61B 2090/571

(57) ABSTRACT

A surgical system for minimally invasive robotic surgery, including a holding arm having a holder for the gripping unit of a conventional endoscope, and a patient-side unit which can be fastened in the vicinity of the patient and allows a defined position of the access to the patient.

### CROSS-REFERENCE TO RELATED APPLICATIONS

This application is the United States national phase of International Patent Application No. PCT/EP2023/070300 filed Jul. 21, 2023, and claims priority to German Patent Application No. 10 2022 118 388.2 filed Jul. 22, 2022, the disclosures of which are hereby incorporated by reference in their entireties.

### BACKGROUND OF THE INVENTION

### Field of the Invention

The invention relates to a surgical system for minimally invasive robotic surgery.

In endoscopy, in particular ureteroscopy (endoscopic procedures in the urethra, bladder, ureter and kidney), endoscopes are used to perform diagnostic tasks (e.g. optical examination of organs) or manipulations (e.g. taking biopsies, removing foreign bodies such as kidney stones) within hollow organs. These can either be rigid (“rigid endoscopes”) or can be angled in at least one degree of freedom (“flexible endoscopes”). During procedures, the endoscopes are typically guided manually by the surgeon, which is technically challenging, especially with flexible endoscopes: The surgeon holds the handle of the flexible endoscope in one hand and uses a lever on the handle to activate the angulation of the endoscope tip and the rotation of the endoscope around its longitudinal axis by turning the handle, while with the other hand, he controls the advance of the flexible endoscope shaft into the patient.

The Avicenna Roboflex is currently a commercially available surgical system for flexible ureteroscopy: The flexible endoscope is docked onto a holding arm located on a cart, which also offers actuation options for angling the endoscope tip and moving the laser fiber.

Intuitive Ion is a surgical system for minimally invasive peripheral lung biopsies that can navigate a flexible bronchoscope telemanipulated through the bronchi of the lung.

Auris Monarch is another surgical system for peripheral bronchoscopy.

Hansen Medical has developed two systems for catheter manipulation, the Magellan Surgical System and the Sensei Surgical System. The Magellan was designed for peripheral, vascular robotic interventions, the Sensei for interventional electrophysiological interventions.

Corindus CorPath GRX is a robotic system for the telemanipulated positioning of catheters in vascular surgery.

### Description of Related Art

A paper by Desai (Desai, Mihir M., et al. “Flexible robotic retrograde renoscopy: description of novel robotic device and preliminary laboratory experience.” Urology 72.1 (2008): 42-46.) describes the use of a specialized robotic catheter system for ureteroscopy in animal experiments on 10 pigs. Here, all renal calices were reached and kidney stones were successfully crushed using a laser fiber.

US 2012/0065470 A1 describes a robotic system for guiding a commercial flexible endoscope, in particular in laryngology. Here, the flexible endoscope is placed in a suitable holder and the actuating element for bending the endoscope tip is placed in a clamp that can activate the actuating element. The entire holder can be rotated around the longitudinal axis of the endoscope and moved along the longitudinal axis of the endoscope using appropriate drives. The drives for the three degrees of freedom are located either in the immediate vicinity of the respective mechanisms or together in a motor housing, with the rotation and angular movement being transmitted via Bowden cables. The robotic system is controlled via a compact control unit with two joysticks (one with one degree of freedom, one with two degrees of freedom), which can either be positioned on a suitable surface or attached to the side rails of the operating table. The robotic system is connected to the side rail of the operating table via a passive stand and roughly positioned.

WO 2013/029 045 A1 describes an endoscope adapter consisting of a holder for the flexible endoscope and a manipulation mechanism which optionally moves the flexible endoscope shaft and/or a tool to be inserted into the working channel of the endoscope in the axial direction. For this purpose, the manipulation mechanism uses rollers, wherein at least one roller is pressed against the endoscope shaft/tool by a spring. It is optionally possible to drive at least one of the two rollers in order to actively control the movement. In one embodiment (FIG. 12), it is proposed to attach the manipulation mechanism to the patient.

WO 2019 139 941 A1 describes an adapter with which a rigid endoscope can be attached to the instrument interface of a medical robot in minimally invasive surgery. This adapter makes it possible to convert the rotation of an output of the instrument drive unit (for faster rotation) or alternatively the rotation of the entire instrument drive unit by a drive in the instrument holder (for slower rotation) into a rotation of the endoscope around its longitudinal axis. It is possible to use various commercially available standalone endoscopes with the adapter (the mounting shells for the endoscope may have to be replaced). Depending on the version, the endoscope is permanently installed in the adapter (FIGS. 3-6) or can be removed from it after opening a latch (FIGS. 8-11; FIGS. 21A-24).

U.S. Pat. No. 10,219,867 B2 describes the Avicenna Roboflex Surgical System: Various commercially available flexible endoscopes can be attached to a holder on the end effector of a robot positioned on a trolley. This holder can be moved back and forth in the direction of the endoscope axis and rotated around the endoscope axis. Furthermore, the holder contains a mechanism for actuating the degree of angular freedom of the flexible endoscope. In addition, mechanisms for activating auxiliary tools (laser fiber, pliers, recovery baskets, etc.) and a pump unit for controlling the flushing of the working channel are available. The system is telemanipulated by the surgeon from a console using two force feedback joysticks (one allows movements back and forth and rotations around the joystick axis, the second has a lever for coarse control of the endoscope angulation), an adjusting wheel for fine control of the endoscope angulation, foot pedals (e.g. for laser fiber and fluoroscopy) and a touch screen. Some safety functions are integrated (laser cannot be fired in the working channel of the endoscope, endoscope is straightened when the laser fiber is inserted), as well as autonomy functions (compensation of the patient's breathing movement by translational movement of the endoscope).

U.S. Past. No. 9,763,741 B2 describes a robotic system for telemanipulation of a flexible endoscope. The endoscope is an instrument specially designed for this robotic system, which is attached to a drive unit that can be positioned by a holding arm. While the robot performs the translation and rotation of the endoscope around its longitudinal axis, the drive unit actuates the bending of the endoscope tip. The flexible endoscope shaft is guided by a rigid sheath guided by a second holding arm, into which sheath the endoscope shaft is inserted.

US 2017/0119412 A1 describes the guidance of a recovery basket by means of holding arms that can be moved under remote control from a surgeon's console or in hands-on mode. If the recovery basket is pulled together to catch an object, the robots automatically adjust the position of the recovery basket so that the object remains in the center of the recovery basket. As soon as the object has been caught, it can be shredded (by laser, fluid or mechanically) and aspirated through a central working channel while still in the basket.

US 2018/0092517 A1 describes a calibration method for flexible endoscopes in which the robotic system moves the endoscope to various target positions and receives feedback on the actual endoscope position via suitable sensors (e.g. electromagnetic sensor systems, cameras, fiber optic sensors). Based on this, correction factors for endoscope actuation are determined and saved. These can depend on various factors (e.g. activated cables, length of the endoscope tip outside the sheath, rotation of the endoscope in relation to the sheath) and can be stored in a calibration matrix. By integrating strain gauges into the instrument drive unit, the cable forces acting on the endoscope can also be monitored.

US 2019/0191967 A1 describes a robotic telemanipulation system with optional haptic feedback, in which the robotic instruments (consisting of holding arm and end effector) and the flexible endoscope for imaging are guided through the working channels of a flexible transport endoscope. The transport endoscope can be attached to a docking station during the surgical procedure. The degrees of freedom of the robotic instruments are activated by a common motor box while an endoscope support system remotely controls insufflation, suction and irrigation of the transport endoscope. The endoscope for imaging and the robotic instruments can be moved as a whole along their longitudinal axis and rotated around their longitudinal axis. To enable precise motion transmission from the motor unit to the robotic instruments, the pretension of the transmitting wire cables can be set automatically at system startup or intraoperatively.

German Patent Application 10 2019 134 352.6 describes a surgical robot for endoscopic applications in very general terms. Here, a flexible endoscope with its handle unit is detachably attached to an instrument base plate, while the movement and the advancement of the shaft is effected by a robotic arm. The degree of angular freedom of the endoscope is controlled by another actuator. Various possible assistance functions are described (gravity compensation, automatic advancement and retraction of the endoscope, automatic change of instruments such as laser fiber and recovery basket, motion compensation for patient movement, automatic orientation of the image, mapping, display of additional functions via augmented reality). The entire system is mounted on a movable platform so as to be mobile.

Manual handling of the endoscope has various disadvantages:
Physical exertion due to the weight of the handle
Unergonomic hand position due to twisting of the handle and/or actuation of the control elements. In some cases, the movement possibilities of the endoscope inside the body are limited by the movement possibilities of the human hand.
Surgeon stands in the area of the X-ray machine during intraoperative X-rays. On the one hand, this makes it necessary to wear a lead vest and, on the other hand, increases the X-ray exposure of the surgeon.
Second surgeon required.
   Confined working conditions, as the available space (typically between the patient's spread legs during ureteroscopic procedures) is limited.
   Complex coordination: When using a tool such as a laser fiber or a retrieval basket, the movements of the endoscope and tool must be coordinated, which requires good coordination between the two surgeons.

The commercially available robotic solutions also have disadvantages:
Pure telemanipulation systems:
   Conversion to manual surgery only possible with difficulty
   Loss of surgical dexterity when moving endoscopes/catheters
   Repeated insertion and removal of the endoscope during stone retrieval in urology requires support from sterile personnel at the operating table

Highly specialized systems:
Surgical systems customized for a specific application =>particularly interesting for large clinics with high treatment numbers
   Apart from Roboflex Avicenna: Use of special robotic instruments, which must be purchased in addition to the manual instruments

The publications mentioned have the following disadvantages with regard to the guidance of flexible endoscopes:

US 2012/0065470 A1: The activated degrees of freedom only allow small movements (especially during translation); for larger movements, the ports on the passive stand must be opened, the system components repositioned and the ports closed again. Intraoperative movement of the endoscope by hand is not easily possible, as removing the endoscope from the holder is complex (opening the holder and opening the clamp for the actuation lever) and there are no corresponding sensors to enable hands-on control of the system.

WO2013/029 045 A1: The roller mechanism shown can actuate the translation of the flexible endoscope shaft or the tool in the working channel of the flexible endoscope shaft. However, the rotation of the endoscope around its longitudinal axis cannot be actuated with the mechanism described. However, this is indispensable, especially for smaller endoscopes that can only be angled in one plane (e.g. ureteroscopes), in order to be able to perform all the desired manipulation tasks inside the patient. Furthermore, it is apparently not possible to quickly release the frictional connection between the endoscope shaft and the rollers, as the rollers are spring-loaded. This means that the surgeon cannot advance the endoscope manually using his dexterity.

The adapter described in WO 2019 139 941 A1 is designed for rigid endoscopes. For this reason, it does not allow an actuation of the endoscope angling. On the other hand, a robot with such an adapter can position and align the handle of a flexible endoscope in space, but not the endoscope tip, as there is no guide for the flexible endoscope shaft and therefore no unique transmission of the movement of the handle to the movement of the endoscope tip.

The Avicenna Roboflex system described in U.S. Pat. No. 10,219,867 B2 has the above-mentioned disadvantages of robotic systems.

The robotic system for interventions with flexible endoscopes described in U.S. Pat. No. 9,763,741 B2, US 2017/0119412 A1 and US 2018/0092517 A1 also has the above-mentioned disadvantages of robotic systems. Furthermore, the use of specialized instruments and the high technical complexity of the system shown (three holding arms required to perform an endoscopic procedure in the kidney) are likely to make it considerably more difficult to use the system economically, particularly in smaller clinics with low case numbers.

Patent US 2019/0191967 A1 focuses on the actuation of the imaging endoscope and the robotic instruments, both of which are only designed for use with this system. The transport endoscope is still controlled manually, and the flexible shaft is only fixed at the position of the handle and patient access.

Compared to German Patent Application 10 2019 134 352.6, the present solution further develops the mechanical design of the system. In particular, the design of the attachment of the endoscope handle to the holding arm, the design of the port attachment at the access to the patient and the possible integration of virtual fixtures to support the surgeon should be mentioned here.

### SUMMARY OF THE INVENTION

It is an object of the invention to provide a surgical system for minimally invasive robotic surgery that enables simplified handling of an endoscope.

According to the invention, the object is achieved by the features as described herein.

The surgical system according to the invention for minimally invasive robotic surgery comprises a holding arm which has a holder for the handle unit of a conventional endoscope. This means that conventional endoscopes can be attached to this holder without any further modifications so that they can be guided by the holding arm. The surgical system also comprises a patient-side unit that can be attached close to the patient. This involves a defined positioning of the patient access.

The advantage of the surgical system according to the invention is that a surgeon can release the handle unit of the endoscope and use his hand, which was originally required for holding the handle unit, for other tasks, for example for guiding the endoscope more precisely. Until now, it has been customary to use a second surgeon for this purpose. This is no longer necessary thanks to the surgical system according to the invention.

Preferably, the surgical system has an output unit for the system status. For robot-assisted solo surgery, the surgeon needs a lot of information, one or more items of which can be displayed on the output unit:
Endoscope image (as little of this as possible should be covered)
Status of additional devices (flushing, laser, X-ray; on the control unit, on the device or on the screen)
Holding arm status

Further possibly interesting information is:
Map of surgery region (on second screen)
World model robot system (on second screen)
Directional instructions for movements of the holding arm

For example, a ring of several individually addressable RGB LEDs on the holding arm can be used as an output unit. This has the following advantages:
Compact and inexpensive
Clearly visible when manipulating the holding arm (this is when the dis^played information is important)
Simultaneous display of system status (=control mode of the robot; via LED color) and direction instructions (via LED activation)
Does not obscure parts of the endoscope image

Furthermore, it is preferred that the holding arm can be positioned manually by an operator and locked in a desired position.

Preferably, it holds its position automatically as soon as the operator no longer exerts any force on the holding arm.

For example, at least one sensor can be provided to detect whether the handle unit is being held by an operator. If this is the case, the control of the holding arm is switched to movement mode so that the holding arm can be moved manually by the operator and brought into a desired position. Its position can then be locked again so that the handle unit of the endoscope is held in the position desired by the surgeon. A plastic cover around the handle unit with integrated capacitive sensors can be used for detection.

Furthermore, it is preferred that the control of the holding arm is designed such that the position of the patient-side unit can be determined by touching the end effector of the holding arm. This is done by determining the position of the end effector via the position of the joint angle of the holding arm, which is known at the time of contact. For example, a probe tip can be attached to the robot-side unit (=end effector attached to the robot arm, into which the endoscope is inserted) only temporarily (e.g. by force closure of a magnet). This allows the endoscope to remain connected to the holding arm during probing.

It is also preferable that the handle unit can be separated from the holding arm without tools, for example by opening the holder. This enables the surgeon to guide the handle unit with one hand as usual in particularly critical situations during an operation.

Furthermore, it is preferred that the control of the holding arm is designed to generate a virtual barrier beyond which the handle unit cannot be moved. This can prevent injury to the patient and/or excessive bending of the endoscope, whereby the known position of the patient-side unit must also be taken into account.

The robot arm can be either a passive arm with brakes and position sensors in the joints or a holding arm with actively driven degrees of freedom. In the case of a robot, it either has integrated torque sensors for detecting external forces or a suitable force/torque sensor on the tool interface. In order to keep the actuation forces low, it is preferable for the holding arm to be fully or partially gravity-compensated.

It is preferable that the endoscope can be inserted into the holder without tools. This can be done using snap locks, magnetic locks, latches attached to the holder or (captive) knurled screws, for example.

A prismatic guide, for example, can be provided to determine the orientation of a port attached to the patient-side unit. To determine the position of the port access, a contact surface can be provided opposite the patient-side unit. As already described, this allows the patient-side unit to be touched or probed with the end effector to determine its position. The stop for probing is preferably detachably connected to the attachment of the endoscope handle unit, for example via a magnet.

An attachment option for the end effectors in the working channel of the flexible endoscope (e.g. laser fiber or retrieval basket) is preferably located in the immediate vicinity of the endoscope handle unit. This should be operable without tools (e.g. click fasteners for attaching the end effectors). It should also make it possible to position the tools preferably in several axes in relation to the endoscope handle, for example to take into account the different hand sizes of different surgeons. Preferably, the fastening option for the end effectors also has an option for fixing mechanical control elements of the end effectors in various positions (e.g. levers for opening and closing the recovery basket).

Preferably, additional control elements operable in a sterile manner are located in the immediate vicinity of the endoscope handle unit in order to activate the flushing of the endoscope and/or the laser and/or to take an X-ray image of the surgical area. These control elements can supplement or replace the foot pedals that were previously used. Their activation requires the surgeon to shift his weight to one leg, which, in conjunction with holding the endoscope handle in a stable position, can lead to unergonomic and physically strenuous postures. The control elements can be designed in various ways, e.g. as toggle switches, pushbuttons, buttons, touchscreens, etc. The following points should be noted:
Sterility: The control elements must be easy to operate even when wearing surgical gloves. In addition, they must either be sterilizable or draped in a sterile manner during intervention. In the second case, it must be possible to operate the device through the sterile drape without any problems.
Compatibility with existing hardware: Preferably, the control elements should be compatible with devices from different manufacturers.
The operating characteristics of the control element must match the device being operated:
   Flushing often runs for a long time, so a control element should be used that maintains the state, e.g. a toggle lever or button. It is also advantageous to be able to immediately recognize the operating status visually, e.g. by the position of a toggle lever, an illuminated button or a status LED.
   The laser should only be triggered while the surgeon is operating the control element. This can be achieved using a push-button, for example. It is also advantageous to be able to immediately recognize the operating status visually, e.g. by an illuminated push-button or a status LED.
   The acquisition of an X-ray image should be triggered once when the surgeon presses the corresponding control element. This can be achieved using a push-button, for example.

The patient-side unit fulfills several tasks:
Defined positioning of patient access: In flexible ureteroscopy, access to the surgical site in the urethra, bladder, ureter or kidney takes place through the patient's urethra. At the beginning of the procedure, a sheath is often placed from the end of the urethra to the surgical site (technically speaking, a tube with a funnel at the entrance) in order to avoid injury to the urinary tract due to repeated advancement and retraction of the ureteroscope. This sheath must be secured against slipping out of the urethra and ureter. Furthermore, the position of this sheath must be known in order to define virtual fixtures to support endoscope insertion into the patient. This can be achieved either via suitable tracking systems or by mechanically fixing the port to the patient-side unit. If the first variant requires an appropriate tracking system (e.g. an external stereo camera or a stereo camera on the patient-side unit), the second variant is easier to implement and therefore preferred. Mechanical locking can be achieved via a force fit and/or form fit, whereby the corresponding mechanism should be operable without tools.
Clamping mechanism shaft: Preferably, the patient-side unit has a clamping mechanism for fixing the flexible endoscope shaft. This allows the surgeon to fix the position of the endoscope shaft and release the endoscope if necessary, for example to move the end effector in the working channel and operate it at the same time (e.g. opening and closing the retrieval basket or activating the laser) or to change the end effector (e.g. from laser fiber to retrieval basket). If the patient-side unit has a clamping mechanism, this can preferably be fixed and released without tools (e.g. using snap locks, magnetic locks or latches). If the patient-side unit has a clamping mechanism, it is also preferable that a sensor system is provided to detect the closing of the clamping mechanism. This can be a limit switch, for example, which is pressed when the clamping mechanism is closed. Alternatively, other sensors such as light barriers, pressure sensors in the clamping surface or time-of-flight sensors can also be used for this purpose. If closing of the clamping mechanism is detected, the range of movement of the holding arm is restricted so that the distance between the endoscope handle and the clamping mechanism cannot be increased any further. This prevents pulling on the endoscope shaft and possible damage to the endoscope as a result.
Defined contact surface for touching the patient-side unit: Unless the attachment of the endoscope handle unit and the patient-side unit are to be provided with markers that are detected by an external tracking system, their position and alignment to each other must be determined in another way. For this purpose, a stop on the attachment of the endoscope handle unit can be used, which is brought into contact with the corresponding geometric features of the sheath and patient-side unit in order to determine the position of the sheath access and the orientation of the sheath. When the user confirms that the desired position has been reached (e.g. by pressing a button or foot pedal), the position and orientation of the port in relation to the holding arm is calculated and saved based on the forward kinematics of the holding arm and the known geometry of the stop and patient-side unit.
Sensors for detecting collisions: If the relative position and alignment of the attachment of the endoscope handle unit and the patient-side unit is not permanently monitored by an external tracking system, but is determined by touch as described above, movements of the patient-side unit can only be detected with the aid of additional sensors. For example, an accelerometer/IMU can be attached to the patient-side unit, which detects its movements. If a movement occurs after the port position and orientation have been saved (e.g. as a result of the user colliding with the patient-side unit), the system can react immediately and, for example, prompt the user to touch the patient-side unit again.
Defined drop site for stone fragments and biopsy material: It is advisable to provide a storage facility for material from inside the patient (in particular stone fragments and biopsy material for ureteroscopy) in the immediate vicinity of the sheath. This is particularly advantageous for larger kidney stones, which have to be broken up by laser and then removed piece by piece from the patient's urinary tract using the retrieval basket. To ensure that the stone fragments or the biopsy material do not get caught on the forceps or the retrieval basket, a blower (compressed air or fan) can also be provided in the area of the drop-off point, which blows the objects in the direction of the drop-off point.

### BRIEF DESCRIPTION OF THE DRAWINGS

The terms Fig., Figs., Figure, and Figures are used interchangeably to refer to the corresponding figures in the drawings.

In the following, preferred embodiments of the invention are described by means of the figures, in which

The Figures show:

FIG. 1: Manual operation of a flexible ureteroscope

FIG. 2: Detailed view of the endoscope tip in the unbent and in the bent state

FIG. 3: Typical end effectors that are integrated into the working channel of flexible ureteroscopes

FIG. 4: Complete setup for the manipulation of flexible endoscopes by individual users in urology with and without visualization of the patient

FIG. 5: Attachment for the endoscope handle unit on the holding arm

FIG. 6: Stop for probing the patient-side unit:

FIG. 7: Mounting for the end effectors

FIG. 8: Sterile manipulable control element on the endoscope handle unit

FIG. 9: General view of the patient-side unit

FIG. 10: Probing of the patient-side unit

FIG. 11: Virtual fixtures to limit the range of movement of the endoscope handle unit

FIG. 12: Use of an LED ring on the holding arm to guide the user into the area of the virtual fixtures

### DESCRIPTION OF THE INVENTION

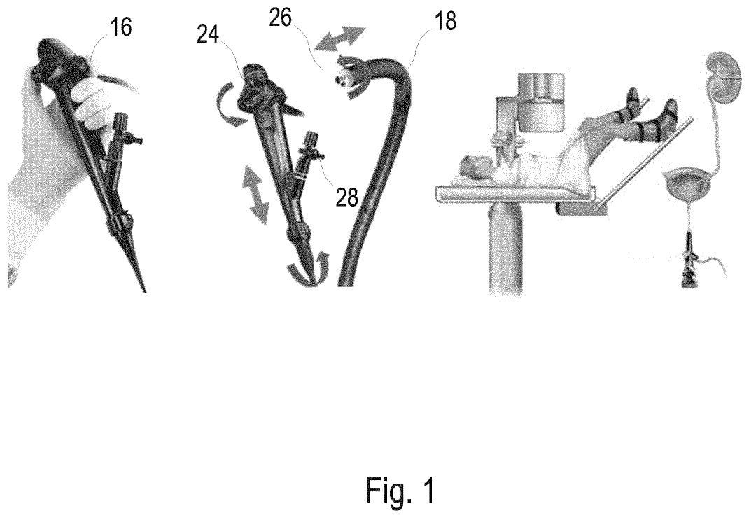

FIG. 1 shows the manual operation of a flexible ureteroscope (endoscope for urological procedures): The physician holds the handle (16) in one hand (left Figure), the other hand guides the flexible shaft (18), usually near the access to the patient. By moving the actuating element (24) (along the left arrow), the endoscope tip (26) can be bent in one plane (right arrow). The plane in which the endoscope tip bends can be varied by rotating the entire endoscope around its longitudinal axis. The endoscope tip is advanced by translating the entire endoscope. Various tools such as an optical fiber for a laser or a retrieval basket for removing kidney stones can be inserted through the working channel (28). Depending on the model of the endoscope, the arrangement and design of the control elements differs; the type and number of endoscope degrees of freedom are typically identical for different endoscopes for one application (e.g. urology).

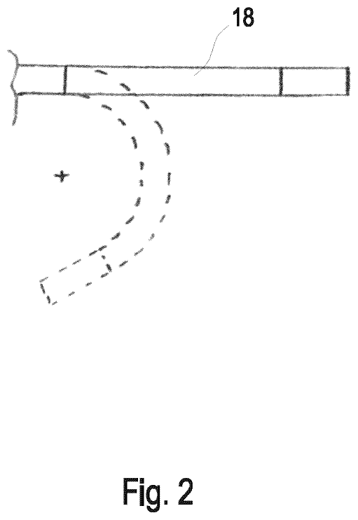

As shown in FIG. 2, the tip of flexible endoscopes for urology can only be bent in one plane and in a certain range due to the small diameter (the endoscope must fit through the patient's urethra): The flexible area of the endoscope tip is bent by actuating the adjusting wheel on the handle using cables/rods running in the endoscope shaft. The endoscope tip and the rest of the shaft remain rigid in the meantime.

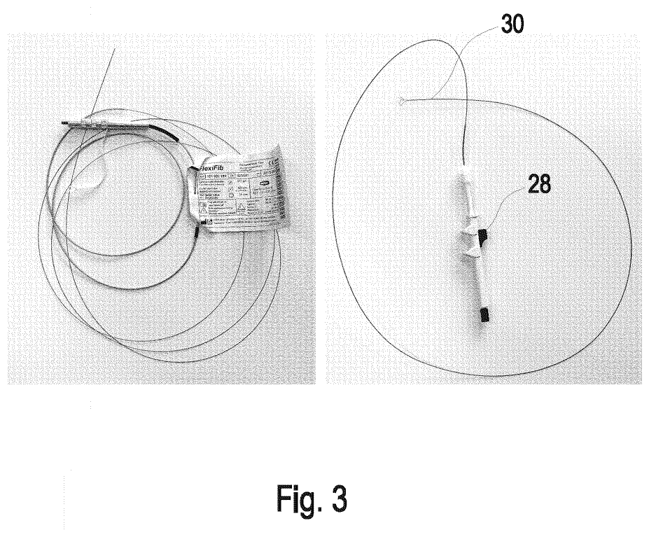

FIG. 3 shows two typical end effectors that are integrated into the working channel of flexible ureteroscopes: Laser fiber for breaking up kidney stones (left), recovery basket for catching the stone debris (right). Translating the black handle element (28) in the direction of the arrow opens and closes the recovery basket (30) at the tip of the end effector.

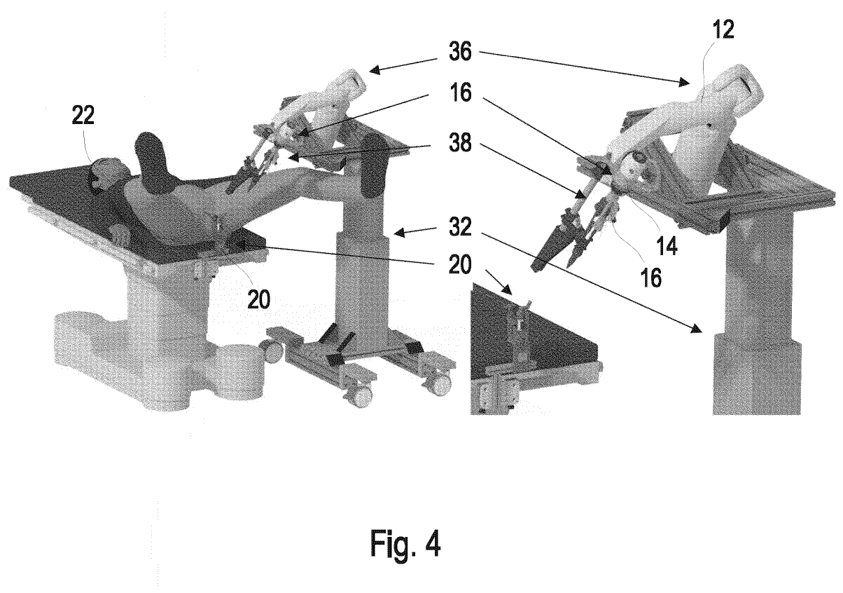

During manual urological procedures with flexible endoscopes (e.g. endoscopic kidney stone removal), the surgeon and assistant are positioned between the patient's legs, which are spread apart, in order to manipulate the flexible endoscope and the tools guided through its working channel. The use of a robot to manipulate the flexible endoscope should make such procedures possible in future without the use of an assistant. FIG. 4 shows a possible overall setup for the manipulation of flexible endoscopes by individual users in urology with (left) and without presentation of the patient (right): The (robotic) holding arm (12) is mounted on a mobile trolley (32), its base being height-adjustable and preferably additionally tiltable in at least one axis. An attachment for the endoscope handle unit (16) is attached to the tool flange of the holding arm (34). The patient-side unit (20) is located in the immediate vicinity of the exit of the patient's urethra. Not shown are the additional computers required to control the robotic support arm (which can be flexibly positioned in the operating room or accommodated in the base of the mobile trolley) and the screen for displaying the endoscope image (which must be visible from the surgeon's position between the patient's legs).

In the overall setup shown, a robot is preferably used as a holding arm. The robotic arm then acts as an intelligent stand for the endoscope handle unit. The surgeon can move and position the handle unit freely in space as usual for manual procedures, with the robotic arm following the movements in a gravity-compensated manner. If the surgeon releases the handle unit or locks a position via a corresponding user input (pressing a button, pressing a foot pedal, voice control, . . . ), the robot is switched to a position-controlled or rigid impedance-controlled mode so that the pose (position and orientation) of the endoscope handle unit is maintained even after it has been released by the surgeon.

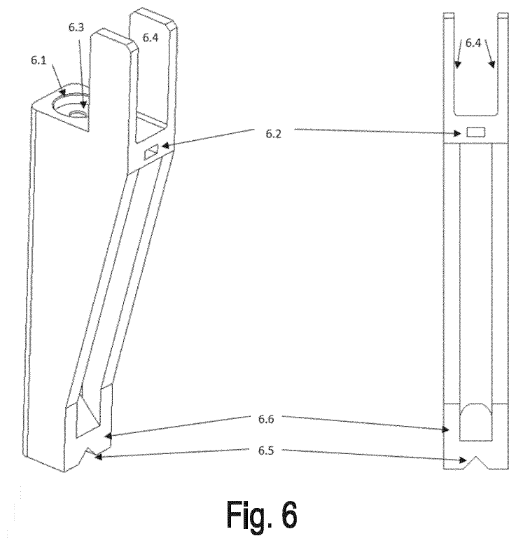

As an alternative to the illustrated attachment to the operating table, the patient-side unit can also be attached to the mobile trolley using an additional support arm. If the degrees of freedom of this additional holding arm have suitable position sensors, the position of the patient-side unit can then be measured by its holding arm instead of touching it via the stop shown in FIG. 6.

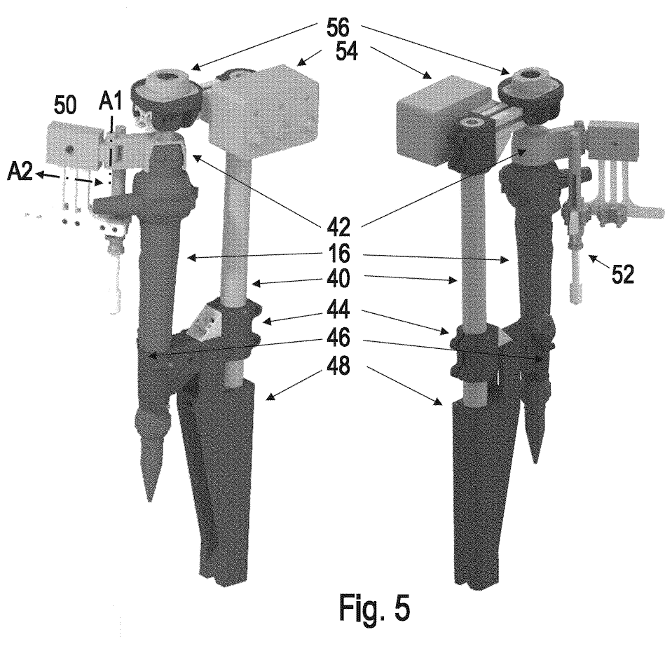

FIG. 5 shows the attachment for the endoscope handle unit on the holding arm The attachment for the endoscope handle unit (16) is connected to the holding arm via a docking interface (56), which is preferably operated without tools. It comprises a mechanical structure (40) to which form-fitting, endoscope-specific holders (42, 44 and 46) are attached. In the example shown, the endoscope is clicked into the upper holder (42), while the lower holder consists of two form-fitting half-shells (44 and 46), which are screwed together. Furthermore, the attachment for the endoscope handle unit has a stop for probing the patient-side unit (48). In addition, an attachment option for the end effectors (50) in the working channel of the flexible endoscope (e.g. laser fiber or retrieval basket) is located in the immediate vicinity of the endoscope handle unit. The example shows the handle unit for a recovery basket (52). Preferably, the arrangement of the fastening options can be adapted to the ergonomic requirements of the system user in several degrees of freedom, for example by rotation around axis A1 or translation along axis A2. In addition, additional control elements (54) operable in a sterile manner are located in the immediate vicinity of the endoscope handle unit in order to activate the flushing of the endoscope and/or the laser and/or to take an X-ray image of the surgical area.

FIG. 6 shows the stop for touching the patient-side unit: The attachment for the endoscope handle unit has a magnet and a conical extension to hold the stop in place. The conical extension engages in the conical recess (6.1) of the stop. The magnet for locking the translation is tightened by a screw, which is screwed into a nut pressed into the recess (6.2) so that its end is located in the bore (6.3). The inner surfaces of the two extensions (6.4) prevent the stop from turning. The stop has a prismatic guide for the port (6.5) at one end and a contact surface opposite the patient-side unit (6.6). If necessary, the stop can have additional shoulders/guide pins to prevent the prismatic guide from twisting around the port

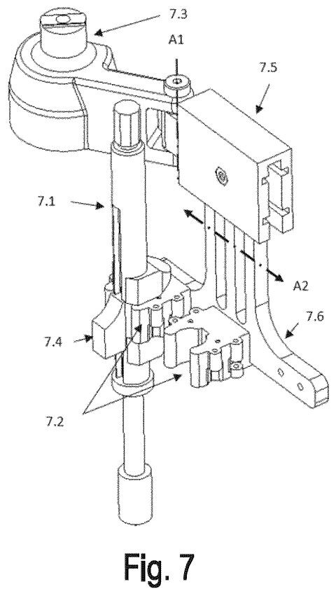

FIG. 7 shows the attachment of the end effectors: The end effectors, such as the handle unit for a retrieval basket (7.1), are fixed in end effector-specific clamps (7.2), which are connected to the attachment for the endoscope handle unit (7.3). Optionally, these clamps can have active elements for interacting with the control elements of the end effectors (not shown), in the case of the recovery basket, for example, to determine the position of the lever for opening and closing the recovery basket (7.4). Preferably, this connection has several lockable degrees of freedom in order to be able to adjust the position of the end effectors to the hand ergonomics of the system user before the start of the operation. In the example, the structural part (7.5) can be rotated about A1 relative to the attachment for the endoscope handle unit (7.3) and the structural part (7.6) can be moved along A2 relative to the structural part (7.5).

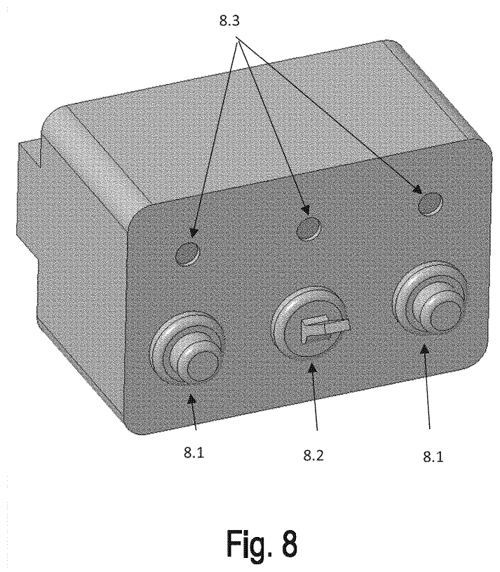

FIG. 8 shows the sterile manipulable control elements on the endoscope handle unit: By attaching the necessary control elements close to the endoscope handle unit to activate the flushing of the endoscope and/or the laser and/or to take an X-ray image of the surgical area, the foot pedals commonly used up to now can be supplemented or replaced. A control element (e.g. a button (8.1) or a switch (8.2)) must be provided for each function. A status display is also preferably provided for each function, e.g. an LED (8.3). The control elements can be read out and the status display activated via a microcontroller, for example.

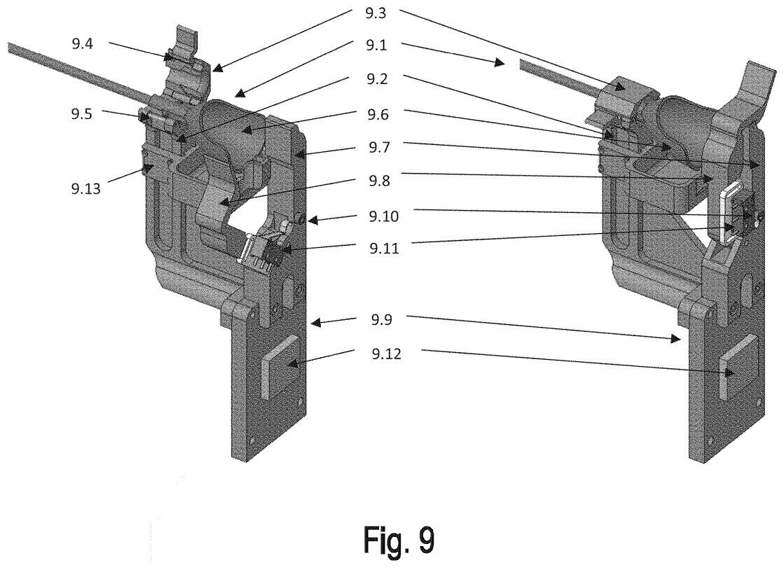

FIG. 9 shows the entire patient-side unit, open on the left and closed on the right: The port (9.1) is inserted into the lower part of the port fastening (9.2), then the upper part of the port fastening (9.3) is closed by means of a latching element (9.4) and a pin (9.5) and the port is fixed in this way. The clamp for the endoscope shaft with the clamp base (9.7) and the clamp lever (9.8) is located in front of the funnel of the port (9.6), which is firmly connected to the lower part of the port attachment via the base plate (9.9). Closing of the terminal is detected by a screw (9.10) pressing the lever of the limit switch (9.11). Optionally, the patient-side unit also has an accelerometer/IMU (9.12), which detects movements of the patient-side unit (e.g. as a result of collisions). In such a case, the user can then be informed that the patient-side unit needs to be probed again. Optionally, the patient-side unit also has a drop point for stone fragments and biopsy material (9.13).

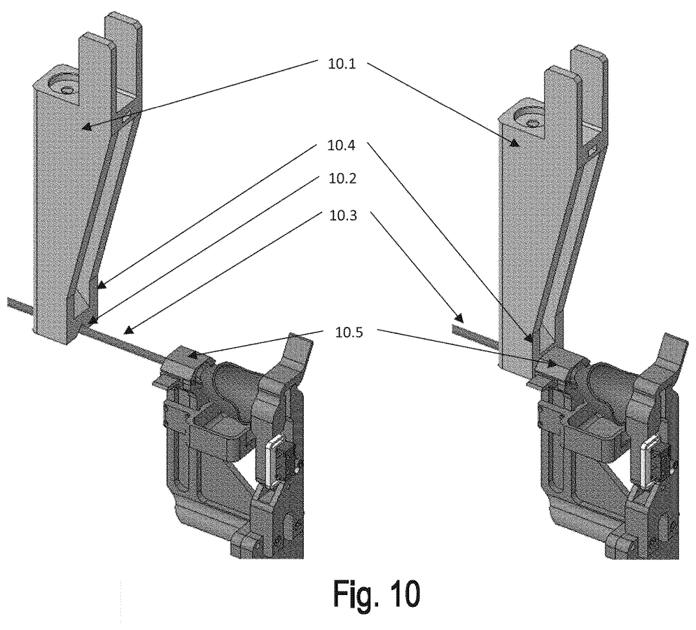

As can be seen in FIG. 10, the patient-side unit is probed in two steps: In the first step (left), the stop (10.1) with its triangular recess (10.2) is placed on the tube of the port (10.3) so that the tube rests against both surfaces of the recess over the entire length of the stop. In a second step (right), the stop is then pushed along the port tube to the port fastening until the contact surface of the stop (10.4) rests against the upper part of the port fastening (10.5).

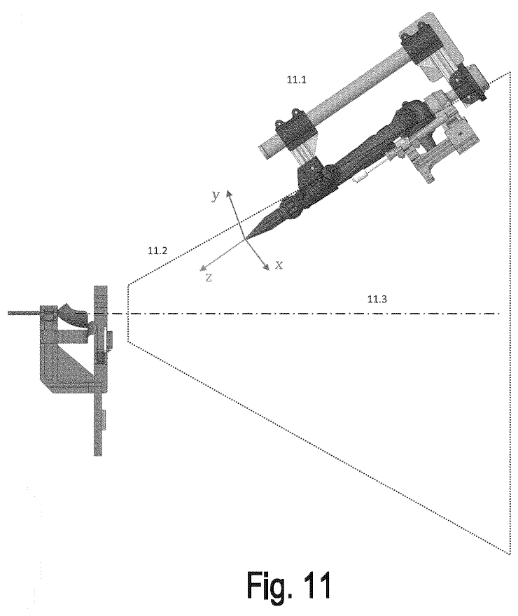

FIG. 11 shows the concept of virtual fixtures for limiting the range of movement of the endoscope handle unit: If the holding arm is a robotic arm or a passive arm with position sensors and brakes in all axes, the range of movement of the endoscope handle unit (11.1) can be limited in order to avoid collisions of the endoscope with the surroundings or damage due to excessive bending of the flexible shaft. For example, as shown, the permissible range of movement of the origin of the coordinate system at the endoscope tip can be limited to a truncated cone in front of the patient-side unit (dotted line 11.2). It is also possible to adjust the alignment of the endoscope longitudinal axis (=z-axis of the drawn coordinate system) to the alignment of the port longitudinal axis (11.3) (for example via a virtual spring-damper system between the coordinate system and the nearest point on the port longitudinal axis).

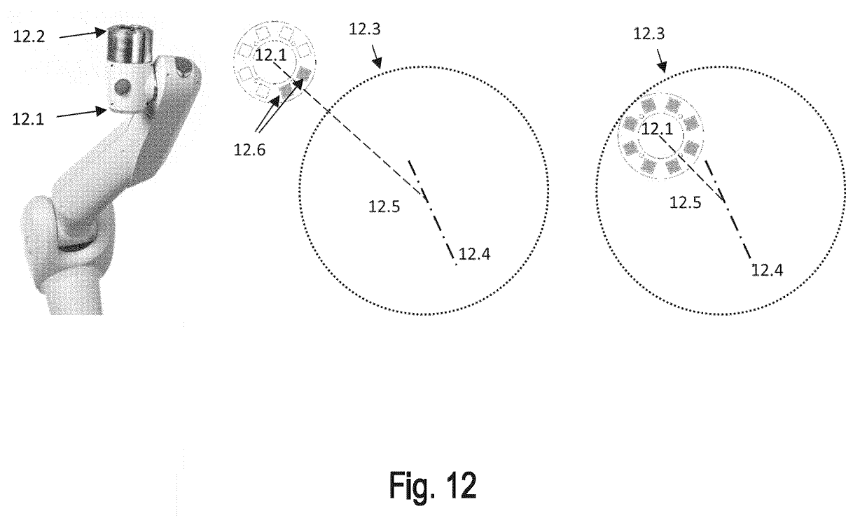

FIG. 12 shows an exemplary concept for the use of an LED ring on the holding arm to guide the user into the area of the virtual fixtures: Preferably, the holding arm has an LED ring with individually controllable LEDs (12.1) near the tool interface (12.2) (see left-hand figure). These can be used to guide the user to the limits of the virtual fixtures (dotted line, 12.3), where safe activation of the fixtures is possible: If the coordinate system at the tip of the endoscope is outside the virtual fixtures (see middle figure), the shortest connection between its origin and the longitudinal axis of the port (dashed line, 12.4) is calculated. The system then calculates how the tool interface of the holding arm must be moved in order to move the endoscope tip along this shortest connection to the longitudinal axis of the lock. The calculated trajectory is projected into the plane of the LED ring (dashed line, 12.5) and the LED(s) closest to this line (12.6) are activated. If the user moves the tool interface of the holding arm in the direction of the illuminated LEDs, the coordinate system on the tip of the endoscope is located within the virtual fixtures at some point (see Figure on the right). The light signal of the LED ring is then changed (e.g. by activating all LEDs) to indicate the possibility of activating the virtual fixtures.

1. A surgical system for minimally invasive robotic surgery comprising:
a holding arm which has a holder for the handle unit of a conventional endoscope,
a patient-side unit that can be attached close to the patient enables defined positioning of the patient access.

2. The surgical system according to claim 1, further comprising an output unit for the system status.

3. The surgical system according to claim 1, wherein the holding arm can be manually positioned by the surgeon and locked in a desired position.

4. The surgical system according to claim 3, wherein the holding arm automatically maintains its position as soon as no more external forces are exerted on it.

5. The surgical system according to claim 1, further comprising at least one sensor for detecting whether the handle unit is held by an operator, wherein, if this is the case, the control of the holding arm is changed to the movement mode.

6. The surgical system according to claim 1, wherein the control of the holding arm is designed such that the position of the patient-side unit can be determined by touching it with the end effector of the holding arm, due to the position of the end effector being known via the position of the joint angles of the holding arm at the time of touching.

7. The surgical system according to claim 1, wherein the handle unit can be separated from the holding arm without tools.

8. The surgical system according to claim 1, wherein the control of the holding arm is designed to generate a virtual barrier beyond which the handle unit cannot be moved, so that injury to the patient and/or excessive kinking of the endoscope is prevented.
